(19) United States
(12) Patent Application Publication
(10) Pub. No.: US 2025/0160768 A1
(43) Pub. Date: May 22, 2025

(54) LOAD BALANCING METHOD FOR A MEDICAL IMAGING DEVICE

(71) Applicant: Siemens Healthineers AG, Forchheim (DE)

(72) Inventor: Rita KRUG, Fuerth (DE)

(73) Assignee: Siemens Healthineers AG, Forchheim (DE)

(21) Appl. No.: 18/947,071

(22) Filed: Nov. 14, 2024

(30) Foreign Application Priority Data
Nov. 17, 2023 (DE) 10 2023 211 486.0

(51) Int. Cl. A61B 6/00

(52) U.S. Cl. CPC A61B 6/44

(57) ABSTRACT

One or more example embodiments relates to a load balancing method for a medical imaging device having a plurality of leveling feet. The method comprises providing a supporting surface with at least one balancing plate; positioning at least one leveling foot on the at least one balancing plate; aligning the medical imaging device relative to the supporting surface by adjusting a height of the at least one leveling foot, wherein the height is adjusted by turning the at least one leveling foot; and readjusting the at least one leveling foot on the at least one balancing plate by applying a predetermined torque, wherein the predetermined torque causes a predetermined load to be achieved on the at least one leveling foot during readjustment.

### CROSS-REFERENCE TO RELATED APPLICATION(S)

The present application claims priority under 35 U.S.C. § 119 to German Patent Application No. 10 2023 211 486.0, filed Nov. 17, 2023, the entire contents of which are incorporated herein by reference.

### FIELD

One or more example embodiments relates to a load balancing method for a medical imaging device. One or more example embodiments further relates to a system for mounting a medical imaging device.

Independent of the grammatical term usage, individuals with male, female or other gender identities are included within the term.

### RELATED ART

A medical imaging device, such as for example a computed tomography apparatus with a gantry, is conventionally positioned standing on a supporting surface. Since the supporting surface is not generally optimally leveled or flat, local high and/or low points of the supporting surface have to be compensated for by adaptable leveling feet. Adjusting the height of the individual leveling feet allows correct alignment of the gantry relative to reference surfaces. Because the gantry has a small degree of residual imbalance and a large mass, which is rotated at high speeds, the medical imaging device may experience undesired natural vibration. The more uniformly is the application force distributed over the leveling feet involved in mounting, the less pronounced is the vibration. In the case in particular of statically overdetermined mounting, it is important to distribute load uniformly since the device may otherwise tip over.

### SUMMARY

To ensure uniform distribution of the application force over the leveling feet, this application force should be measured when the imaging device is installed. However, this is technically complex and difficult to perform without changing the installation state, and is therefore not generally done.

One or more example embodiments provides a method for mounting a medical imaging device, such as a computed tomography apparatus with a gantry, so improving the vibration behavior of the device during operation.

### BRIEF DESCRIPTION OF THE DRAWINGS

Example embodiments are explained below with reference to the appended figures. The depiction in the figures is schematic, highly simplified and not necessarily true to scale. In the figures:

FIG. 1 shows a sequence diagram for method for balancing loads of a medical imaging device,

FIG. 2 shows a portion of a system for mounting the medical imaging device, and

FIG. 3 is a plan view onto a system for mounting the medical imaging device.

### DETAILED DESCRIPTION

One or more example embodiments relates to a load balancing method for a medical imaging device, such as a gantry of a computed tomography apparatus, wherein the medical imaging device has a plurality of leveling feet. The method comprises the following steps: providing the supporting surface with at least one balancing plate, positioning at least one leveling foot on the at least one balancing plate, adjusting the height of at least one of the leveling feet to align, in particular level, the medical imaging device relative to the supporting surface, and readjusting the at least one leveling foot on the at least one balancing plate by applying a predetermined torque, wherein the predetermined torque is sufficiently great for a predetermined load to be achieved on the at least one leveling foot during readjustment.

This makes it possible to level out locally different roughness values and locally different unevennesses of the supporting surface using the at least one balancing plate. The leveling feet have a pitch thread, for example, with which they are screwed into the medical imaging device, so as to enable height adjustment of the medical imaging device. Leveling out of the roughness of the supporting surface results in known, constant frictional behavior between the at least one leveling foot and the balancing plate beneath it. It is thus possible to draw a conclusion as to the load on this leveling foot from the torque applied to the leveling foot. A balanced load on the at least one leveling foot can consequently be readjusted using a specified tightening torque of the leveling foot.

Readjustment of the (single) leveling foot is sufficient, in particular, to balance the load distribution over the further leveling feet and thus the entire medical imaging device. In particular, it is possible (with known geometry or mass distribution of the medical device) to use the application force (also known as load) on one leveling foot to calculate the application force on the other leveling feet. It is thus possible to distribute the load uniformly or in a defined manner over the leveling feet when installing the medical imaging device, such that the rotating gantry's considerable mass excites less vibration in the medical imaging device.

The balancing plate provides a supporting surface of defined roughness beneath at least one leveling foot. It is also conceivable to arrange the or multiple balancing plate(s) beneath multiple leveling feet, in particular beneath all of the plurality of leveling feet. In this way, multiple leveling feet of the medical imaging device can be readjusted at their various positions. The balancing plate is preferably not deformable, so providing a maximally rigid surface with constant coefficients of friction. It is also conceivable for the balancing plate to be of two-part configuration. In other words, the balancing plate may have a bottom and a top, the two sides differing from one another. The bottom may be more adaptable and/or softer relative to the supporting surface, while the top may be more rigid. This results in a balancing plate which is both adaptable and particularly flat.

At least one of the two sides of the balancing plate, in particular the top of the balancing plate, may have a constant roughness value at least in a sub-region. In other words, at least one side may have a uniform coefficient of friction, preferably in the middle of the surface. This may result in equal friction forces, which are defined as the product of the normal force on the side and the coefficient of friction, preferably in the middle of the surface of the balancing plate.

The bottom may moreover be of non-slip configuration, with the bottom thus adhering to the supporting surface. The balancing plate may also be of one-piece configuration, in particular having the same properties on both sides. This is advantageous with regard to manufacture of the balancing plate, in particular as an injection molding. The balancing plate may take the form of a planar plate. In other words, the extent of the balancing plate in two spatial directions may be distinctly greater than in a third spatial direction orthogonal thereto. The balancing plate may for example have side dimensions of between 5 cm and 30 cm, preferably 8 cm to 15 cm, and a thickness of 0.3 to 10 mm, preferably 1 mm to 2 mm.

The leveling feet of the medical imaging device are height-adjustable, such that the alignment of the device can be readjusted. To this end, there are preferably mating threads between in each case one leveling foot and the medical imaging device, in particular a pitch thread. A plurality of (for example 3 to 6, in particular 4 or 5) leveling feet enable the load of the device to be distributed over multiple leveling feet. The device is positioned with at least one leveling foot on the at least one balancing plate. This may expediently be the least heavily loaded leveling foot. Readjustment of the leveling foot results in friction both between the mating threads and between the bottom of the leveling foot and the supporting surface or balancing plate located beneath it. The thread-related friction, in particular the frictional force, between the leveling foot and the medical imaging device is known, for example is ascertained by testing. Likewise, the frictional force between the bottom of the leveling foot and the top of the balancing plate is known, for example ascertained by testing. A defined torque may then be applied using a torque wrench, which defined torque, minus the frictional forces, results in a predetermined application force at the readjusted leveling foot. This leads to a defined application force distribution and consequently low-vibration installation of the gantry.

Since the friction forces, in particular resistance forces, arising during readjustment are known and reproducible, a constant ratio is obtained between a torque during readjustment of the leveling foot on the balancing plate and an application force on this leveling foot. This torque can be ascertained experimentally prior to readjustment. In particular, a relationship may be established between torque and application force, this relationship stemming from constant friction ratios. Consequently, the installation plate may achieve the objective of keeping constant the frictional behavior of the at least one installation foot on its supporting surface, the balancing plate.

According to one embodiment, the at least one balancing plate may be attached non-rotatably to the supporting surface, in particular adhesively bonded and/or bolted to the supporting surface. The balancing plate may, moreover, also have an adhesive surface on its bottom. Furthermore, the balancing plate may have holes for accommodating assembly means, such as bolts, for anchoring purposes. The balancing plate may additionally or alternatively also be attached in another way to the supporting surface and/or be attached to the supporting surface by a combination of multiple fastening options. In this way, the balancing plate may be non-displaceable or unshiftable, in particular fixedly mounted, after assembly with the supporting surface. Furthermore, the balancing plate may thereby be loaded with loads, in particular a torque, orthogonally to a surface normal of the supporting surface, without slipping, bending or splitting apart.

The balancing plate may for example be made from metal or plastic material. It may have a coating with defined frictional properties, for example a paint finish.

According to one alternative embodiment, the balancing plate may be configured as a coating, in particular a curing liquid coating, on the supporting surface. The coating may be poured onto the floor as a leveling layer, distributed uniformly and cured. Epoxy resins are particularly suitable for this purpose since they can provide a uniformly smooth surface after curing. Curing rubber coatings are also conceivable. The coating may produce the advantage of leveling large regions of the supporting surface particularly efficiently and providing them with a substantially even roughness.

According to one embodiment, the balancing plate may reduce, in particular completely level out, supporting surface unevennesses. At least the bottom of the balancing plate may take the form of an adaptable surface. In this way, the balancing plate can compensate for local unevennesses.

According to one embodiment, the medical imaging device may be statically overdetermined due to the plurality of leveling feet. The base of the device may be regarded roughly as a plane which requires three supporting elements to be completely statically determined in space. The medical imaging device may comprise four or more leveling feet, so rendering the medical imaging device statically overdetermined. This allows the total load of the medical imaging device to be introduced particularly uniformly into the supporting surface.

According to one embodiment, the at least one leveling foot may have at least one plunger, wherein the plunger may comprise a pitch thread and wherein the leveling foot may be height-adjusted by turning the pitch thread of the plunger in a complementary internal thread in the medical imaging device. The pitch thread of the plunger may be a metric thread, but in particular also a different type of thread. The pitch thread is arranged at an end distal to the leveling foot plate. The plunger may be configured for screwing into the internal thread of the medical imaging device. So that the internal thread of the medical imaging device may be of complementary configuration relative to the pitch thread of the plunger, both complementary right- and left-hand threads may be provided.

A load acting on a leveling foot is preferably at least substantially proportional to a torque needed to turn the leveling foot. In other words, the proportion of the load of the leveling foot relative to the total load of the device increases as the torque increases which is being used to wind the height-adjustable leveling foot further out from the bottom of the medical imaging device. This enables turning of the leveling foot to vary the load that is applied to the leveling foot. Furthermore, readjustment of one leveling foot can lead to redistribution of the respective loads over other leveling feet of the device. This may result in a uniform load distribution.

The frictional behavior between the mating pitch thread and the complementary internal thread is preferably known. The coefficient of friction of the mating threads can be established by experimental testing. This allows a relationship to be ascertained between the supporting element load acting along a displacement direction of the mating threads and a torque with which the mating threads are wound into one another. This relationship may be linear. Because the coefficients of friction may be constant, a uniform relationship can be expected as the mating threads are repeatedly brought together.

The frictional behavior between a bottom of the leveling foot and the balancing plate located beneath it is preferably known. The bottom of the leveling foot may be formed at least in part by the leveling foot plate. The load to be applied during readjustment of the at least one leveling foot is dependent on the torque with which the at least one leveling foot is turned relative to the installation plate. In order to obtain experimentally ascertained torque values, the frictional behavior between the bottom of the leveling foot and the leveling foot located beneath it has in particular to be constant.

The predetermined torque may preferably be determined taking account of the known frictional behavior of the mating threads and/or the frictional behavior between a bottom of the leveling foot and the balancing plate located beneath it. This can yield the advantage that experimentally determined torques for turning at least the one leveling foot always result in constant corresponding loads.

The leveling foot plate and the plunger of the leveling foot may preferably be joined together for rotation. This specifically prevents any relative motion, in particular rotation, between the two components of the leveling foot. As a consequence, the accuracy of the method can be increased, as no coefficients of friction need to be taken into account between said components. Moreover, the plunger may be made from a metallic material, in particular steel, preferably tool steel. The plunger and the leveling foot plate may be of one-piece configuration, in particular consist of the same material.

The leveling foot may preferably be of multipart configuration, in particular at least in part have a coating, and/or at least in part be formed from a second material. The second material may comprise an elastomer, preferably rubber. Furthermore, the second material may be configured to be a particularly low-friction friction partner for the at least one balancing plate. In this way, readjustment may proceed with reduced expenditure of force, in particular reduced torque. The leveling foot plate of the leveling foot is in particular formed of the second material. It is also conceivable for the leveling foot plate to have a coating which is formed of the second material. Furthermore, the leveling foot plate may be of one-piece configuration with the plunger and have an insert of a second material, which is inserted into the leveling foot plate, in particular adhesively bonded or otherwise fastened therein. The second material may furthermore be of particularly vibration-absorbent, in particular flexible, configuration. In this way, the leveling foot plate may damp periodic motion, in particular vibration, of the medical imaging device.

The coefficient of friction between pitch thread and internal thread may preferably be reduced with a lubricant, in particular oil. Reducing the friction within the mating threads of the plurality of leveling feet reduces the torque needed for readjustment of the at least one leveling foot.

The leveling foot may preferably have a hole which extends in particular centrally along the plunger. The hole may be configured to accommodate a fastening means (e.g., screw, bolt, etc.). In this way, the leveling foot may be anchored to the supporting surface. This may consequently increase the tolerance of the device to vibration.

The leveling foot may preferably have an actuating face, in particular an actuating region, so that it can be turned by a driver tool, in particular a torque wrench. The actuating face may be arranged at a distal end of the plunger. By turning the leveling foot at the actuating region, the leveling foot is turned relative to the supporting surface, in particular the balancing plate, and necessarily displaced relatively along a longitudinal direction, in particular is wound out of the internal thread of the medical imaging device. The actuating face may be configured as a polyhedron and/or hexagon, in particular Allen head, such that a corresponding tool may engage therewith. The actuating region may lie inside the medical imaging device and be reachable. Additionally or alternatively, the actuating face may be arranged on the outer circumference of the leveling foot plate. This means that the actuating region is accessible outside the medical imaging device.

One or more example embodiments further relates to a system for mounting a medical imaging device, in particular a gantry of a computed tomography apparatus. The system comprises a plurality of leveling feet, which are configured to be screwed into the medical imaging device, and at least one balancing plate, which can be and/or is attached to a supporting surface, wherein at least one of the leveling feet can be and/or is arranged on the at least one balancing plate, wherein a given torque may be applied to the at least one leveling foot relative to the medical imaging device in such a way that the load of the medical imaging device is distributed uniformly over the plurality of leveling feet.

The system may in particular comprise three, four or five, or indeed optionally more leveling feet. This enables the medical imaging device to be mounted in statically overdetermined manner. The leveling feet may be screwed height-adjustable into the medical imaging device using mating threads for each leveling foot. A balancing plate may be positioned beneath at least one leveling foot. In this way, the friction partners may be defined between the at least one leveling foot and its supporting surface, the balancing plate. In other words, the frictional behavior between the at least one installation foot and the balancing plate is constant or repeatable. The at least one leveling foot may have a specific torque applied to it relative to the two friction partners a) mating threads and b) friction between leveling foot and balancing plate, whereby a proportion of the load borne by the at least one leveling foot may be varied, in particular increased. The proportion of the load borne by the further leveling feet is also necessarily varied thereby, such that a balanced load distribution can be achieved over the plurality of leveling feet of the medical imaging device.

One or more example embodiments further relates to a medical imaging device having a system according to one or more example embodiments. Load distribution for the medical imaging device may thus be achieved in the manner according to one or more example embodiments. One or more example embodiments further relates to a medical imaging device which is configured to be installed using a method according to one or more example embodiments.

Individual embodiments and individual features may be combined with other embodiments and other features and so form new embodiments. The configurations and advantages of the embodiments and the features also apply analogously to the new embodiments. Furthermore, configurations and advantages mentioned in relation to the medical imaging device also apply analogously to the method and vice versa.

In the figures identical features are denoted with the same reference characters.

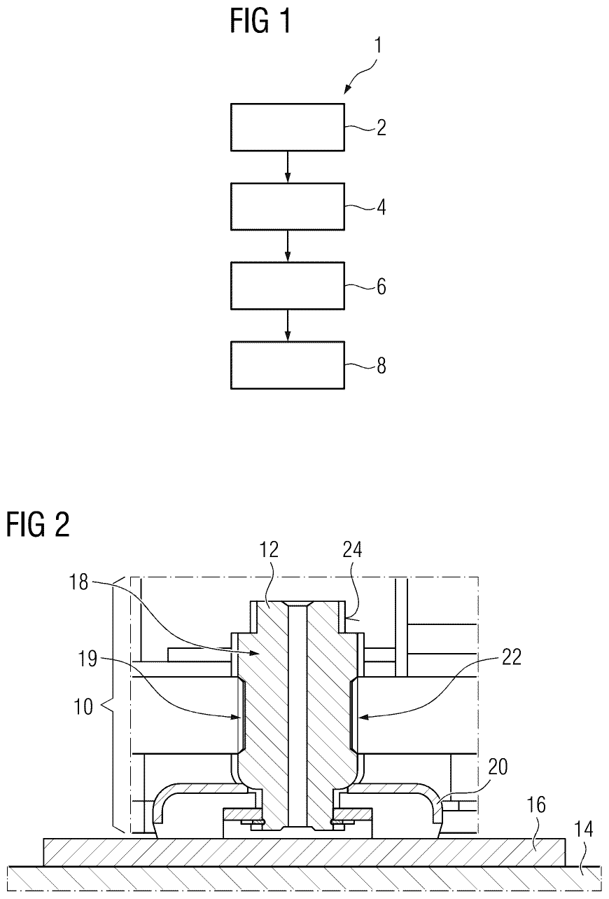

FIG. 1 shows the sequence of individual steps of the method 1. A first step involves providing 2 a supporting surface 14 with a balancing plate 16. The balancing plate 16 provides a planar surface with constant coefficients of friction over at least one part of, in particular the entire, surface of the balancing plate 16. A second step involves positioning 4 a leveling foot 12 of the medical imaging device 10 on the balancing plate 16. The medical imaging device 10 comprises a plurality of leveling feet 12, in particular 3, 4 or 5 leveling feet 12. The medical imaging device 10 is then aligned 6 relative to the supporting surface 14 by height adjustment of at least one of the leveling feet 12.

The leveling feet 12 are configured to move in and out of the medical device when said leveling feet 12 are turned relative to the medical imaging device 10. In this way, the distance between a leveling foot plate 20 of a leveling foot 12 and the medical imaging device may be varied. Unevennesses of the supporting surface 14 may consequently be compensated for using the height-adjustable leveling feet 12. Alignment 6 may be checked and established in space relative to various reference surfaces on a gantry (e.g. stationary frame, drum). After alignment 6, it may be the case that the distribution of application forces over the individual leveling feet 12 is uneven due to the position of the center of mass relative to the respective leveling feet 12. On rotation of the gantry, its considerable mass may excite vibration of the medical imaging device.

The at least one leveling foot is therefore readjusted 8 on the at least one balancing plate. In particular, it is sufficient to readjust just one of the plurality of leveling feet 12, so as to balance the load distribution of the entire medical imaging device 10 over all the loaded leveling feet 12. In doing this, a predetermined torque is applied to the leveling foot 12. In other words, the leveling foot 12 is wound out of the medical imaging device 10, in particular out thereof against the balancing plate 16, using the predetermined torque. Owing to a known frictional behavior between the one leveling foot 12 and the balancing plate 16 and additionally a known frictional behavior of mating threads consisting of a pitch thread 19 of the leveling foot 12 and an internal thread 22 of the medical imaging device 10, the load on the one leveling foot 12 is readjustable with the predetermined torque. This enables a predetermined load on the one leveling foot 12 to be established. It also in particular enables distribution to other leveling feet 12 to be balanced, so optimizing the vibration behavior of the medical imaging device 10.

FIG. 2 shows a portion of the system for mounting the medical imaging device 10. A balancing plate 16 is arranged on a supporting surface 14. The balancing plate 16 may be adhesively bonded and/or bolted and/or attached in some other way to the supporting surface 14 in such a way that it cannot slip or be distorted or split apart relative to the supporting surface 14. A leveling foot 12 is positioned fitted above the balancing plate 16. The leveling foot comprises a plunger 18, on which a leveling foot plate 20 is arranged in such a way that the leveling foot plate 20 is in contact with the balancing plate 16. The plunger 18 may comprise a metallic tool steel and be formed integrally, in particular in one piece, with the leveling foot plate 20. The leveling foot plate 20 may comprise an elastic material, preferably rubber, in particular a second material. The leveling foot plate 20 is in particular coated with the second material. It is also conceivable for the leveling foot plate 20 to be formed of the second material, said second material differing from the material of the leveling foot 12. This can bring about enhanced damping behavior and simultaneously make the leveling foot 12 stronger. At an opposing, distal end, the leveling foot 12 has an actuating face 24, in particular an actuating region, which is configured to engage with a complementary tool, in particular a torque wrench. In this way, the leveling foot 12 can advantageously be turned relative to the medical imaging device 10. The medical imaging device 10 comprises an internal thread for each leveling foot 12, said thread being complementary in configuration to a pitch thread 19 formed on the outer circumference of the plunger 18. The leveling foot 12 can consequently be screwed into the internal thread 22 of the medical imaging device 10. Furthermore, the plunger 18 may have a central hole which extends along a longitudinal axis of the plunger. In this way, the entire leveling foot 12 can be anchored in the supporting surface 14 by way of a fastening means.

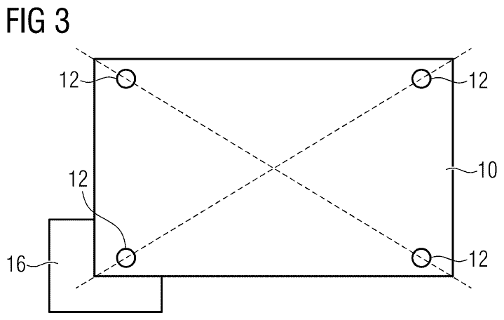

FIG. 3 shows a plan view onto a system for mounting the medical imaging device 10. A bottom of the medical imaging device 10, shown in FIG. 3, has four leveling feet 12. This enables the system to be mounted in statically overdetermined manner. Consequently, different loads on the individual leveling feet 12 may lead to tipping, in particular over the two diagonals depicted. A balancing plate 16 is arranged between supporting surface 14 and one of the leveling feet. The leveling foot 12 positioned thereon may be turned in such a way with a specified torque that the load thereon reaches a predetermined value. The torque to be applied should not lead to it being possible for the leveling foot 12 to be turned on the balancing plate 16, and therefore the leveling foot 12 has initially to be wound into the medical imaging device 10 and then wound back out using the torque.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A load balancing method for a medical imaging device, the method comprising:
providing a supporting surface with at least one balancing plate;
positioning at least one leveling foot on the at least one balancing plate;
aligning the medical imaging device relative to the supporting surface by adjusting a height of the at least one leveling foot, wherein the height is adjusted by turning the at least one leveling foot; and
readjusting the at least one leveling foot on the at least one balancing plate by applying a predetermined torque, wherein the predetermined torque causes a predetermined load to be achieved on the at least one leveling foot during readjustment.

2. The method of claim 1, wherein the at least one balancing plate is attached non-rotatably to the supporting surface.

3. The method of claim 1, wherein the balancing plate is configured as a coating on the supporting surface.

4. The method of claim 1, wherein the at least one leveling foot includes at least one plunger, and the at least one plunger comprises a pitch thread and the at least one leveling foot is height-adjusted by turning the pitch thread of the plunger in a complementary internal thread in the medical imaging device.

5. The method of claim 1, wherein a load acting on the at least one leveling foot is at least substantially proportional to a torque required to turn the at least one leveling foot.

6. The method of claim 1, wherein a frictional behavior between a bottom of the at least one leveling foot and the balancing plate is known, the balancing plate being beneath the bottom of the at least one leveling foot is known.

7. The method of claim 1, wherein the predetermined torque is based on at least one of a known frictional behavior of mating threads or a frictional behavior between a bottom of the at least one leveling foot and the balancing plate, the balancing plate being under the at least one leveling foot.

8. The method of claim 4, wherein the at least one leveling foot plate and the at least one plunger of the at least one leveling foot are joined together for rotation.

9. The method of claim 1, wherein the at least one leveling foot is a multipart configuration.

10. The method of claim 7, wherein the coefficient of friction of the mating threads is reduced with a lubricant.

11. The method of claim 4, wherein the at least one leveling foot has a hole which extends along the plunger.

12. The method of claim 1, wherein the at least one leveling foot has an actuating face, in particular an actuating region, so that it can be turned by a driver tool, in particular a torque wrench.

13. A system for mounting a medical imaging device, the system comprising:
a plurality of leveling feet, the plurality of leveling feet configured to be screwed into the medical imaging device, and
at least one balancing plate, the at least one balancing plate being attachable or attached to a supporting surface, wherein at least one of the leveling feet is arrangeable or arranged on the at least one balancing plate, and a given torque may be applied to the at least one leveling foot relative to the medical imaging device such that the load of the medical imaging device is distributed uniformly over the plurality of leveling feet.

14. A medical imaging device having the system of claim 13.

15. A medical imaging device which is configured to be installed using the method of claim 1.

16. The method as claimed in claim 2, wherein the at least one balancing plate is at least one of adhesively bonded or bolted to the supporting surface.

17. The method of claim 3, wherein the balancing plate is configured as a curing liquid coating on the supporting surface.

18. The method of claim 9, wherein the multipart configuration includes at least in part at least one of a coating or a second material.

19. The method of claim 11, wherein the hole which extends centrally along the at least one plunger.
